(19) United States
(12) Patent Application Publication
(10) Pub. No.: US 2026/0045322 A1
(43) Pub. Date: Feb. 12, 2026

(54) CLINICO-OMICS DATA ASSISTANT

(71) Applicant: DNAnexus Inc., Mountain View, CA (US)

(72) Inventors: Mengtian Zhang, Sunnyvale, CA (US); Georgios Asimenos, Las Vegas, NV (US); Jeffrey Wiser, Brookeville, MD (US); Marek Smid, Prague (CZ); Zuzana Odstrcilova, Prague (CZ); Lucie Stanek Merunkova, Prague (CZ); Josef Strunc, Prague (CZ)

(21) Appl. No.: 19/292,111

(22) Filed: Aug. 6, 2025

Related U.S. Application Data
(60) Provisional application No. 63/679,929, filed on Aug. 6, 2024.

(51) Int. Cl. G16B 50/20; G16B 30/10; G16B 50/10

(52) U.S. Cl. CPC G16B 50/20; G16B 30/10; G16B 50/10

(57) ABSTRACT

The subject technology receives a natural language query from a user related to a clinico-omics data analysis. The subject technology performs a context concatenation function combining different information sources to generate an input for a large language model (LLM) agent. The subject technology processes the natural language query through the LLM agent using at least the input. The subject technology determines whether additional information is needed after processing the natural language query. The subject technology performs a tool execution loop when it is determined that additional information is needed. The subject technology iteratively repeats the tool execution loop until reaching a satisfactory answer or predetermined tool call limit. The subject technology generates, after completing the tool execution loop, a final answer and a set of cohorts including a first set of associated SQL queries, a second set of statistical summaries, and a third set of visualization charts.

### CROSS-REFERENCE TO RELATED APPLICATIONS

This application claims priority to U.S. Provisional Patent Application No. 63/679,929, filed on Aug. 6, 2024, entitled “DATA MANAGEMENT PLATFORM,” and the contents of which are incorporated herein by reference in its entirety for all purposes.

### BACKGROUND

The field of biomedical research increasingly relies on the analysis of large-scale clinico-omics datasets that combine clinical phenotypic data with molecular omics data, including genomic, transcriptomic, proteomic, and metabolomic information. Existing data management platforms can require users to have specialized technical knowledge of database query languages and complex data structures to effectively explore and analyze these datasets. Current systems can expose users to raw SQL queries and require understanding of cryptic field names and database schemas, creating significant barriers for non-technical researchers who need to perform sophisticated data analysis.

### TECHNICAL FIELD

The subject matter disclosed herein relates generally to data management and analysis systems for biomedical research, and more specifically to intelligent data processing platforms that utilize artificial intelligence technologies to facilitate the exploration and analysis of complex clinico-omics datasets.

### BRIEF DESCRIPTION OF THE DRAWINGS

Some examples are shown for purposes of illustration, and not limitation, in the figures of the accompanying drawings. In the drawings, which are not necessarily drawn to scale, like numerals may describe similar components in different views or examples. To identify the discussion of any particular element or act more easily, the most significant digit or digits in a reference number refer to the figure number in which that element is first introduced.

FIG. 1 is a diagrammatic representation of a networked computing environment in which some examples of the present disclosure may be implemented or deployed.

FIG. 2 illustrates a user interface for a clinico-omics data assistant that enables exploring clinico-omics datasets, in accordance with an embodiment of the subject technology.

FIG. 3 illustrates a user interface for the clinico-omics data assistant, in accordance with an embodiment of the subject technology.

FIG. 4 illustrates an example of a user interface, in accordance with an embodiment of the subject technology.

FIG. 5 illustrates an example of a user interface that includes a combination of various graphical elements, in accordance with an embodiment of the subject technology.

FIG. 6 illustrates an example of a backend architecture, in accordance with an embodiment of the subject technology.

FIG. 7 illustrates examples of a user question and a correct answer generated by the clinico-omics data assistant, in accordance with an embodiment of the subject technology.

FIG. 8 illustrates an example of an application architecture, in accordance with an embodiment of the subject technology.

FIG. 9 is a flow diagram illustrating operations of a system (e.g., backend architecture) in performing a method, in accordance with some embodiments of the present disclosure.

FIG. 10 illustrates a machine-learning pipeline, according to some examples.

FIG. 11 illustrates training and use of a machine-learning program, according to some examples.

FIG. 12 illustrates an aspect of the subject matter in accordance with one embodiment.

### DETAILED DESCRIPTION

Reference will now be made in detail to specific example embodiments for carrying out the inventive subject matter. Examples of these specific embodiments are illustrated in the accompanying drawings, and specific details are set forth in the following description to provide a thorough understanding of the subject matter. It will be understood that these examples are not intended to limit the scope of the claims to the illustrated embodiments. On the contrary, they are intended to cover such alternatives, modifications, and equivalents as may be included within the scope of the disclosure.

While existing platforms may provide basic cohort browsing capabilities through structured interfaces with expandable data dictionaries and statistical visualizations, they lack the ability to interpret natural language queries or provide intelligent assistance for complex data exploration tasks. The complexity of clinico-omics data, which often involves multi-entity datasets with longitudinal information, genomic variants, gene expression data, and clinical metadata, presents particular challenges for researchers who need to identify patient cohorts based on sophisticated criteria combining phenotypic and molecular characteristics. Embodiments of the subject technology provide an intelligent data management platform that democratizes access to complex biomedical datasets by providing natural language query capabilities, automated SQL generation, and contextual assistance while maintaining the underlying technical sophistication required for accurate scientific analysis.

The subject technology provides advantages over existing biomedical data platforms by eliminating the need for users to possess specialized technical knowledge of database query languages and complex data structures. Unlike other systems that expose users to raw SQL queries and require understanding of cryptic field names and database schemas, the subject technology enables non-technical researchers to perform sophisticated data analysis tasks through natural language interfaces.

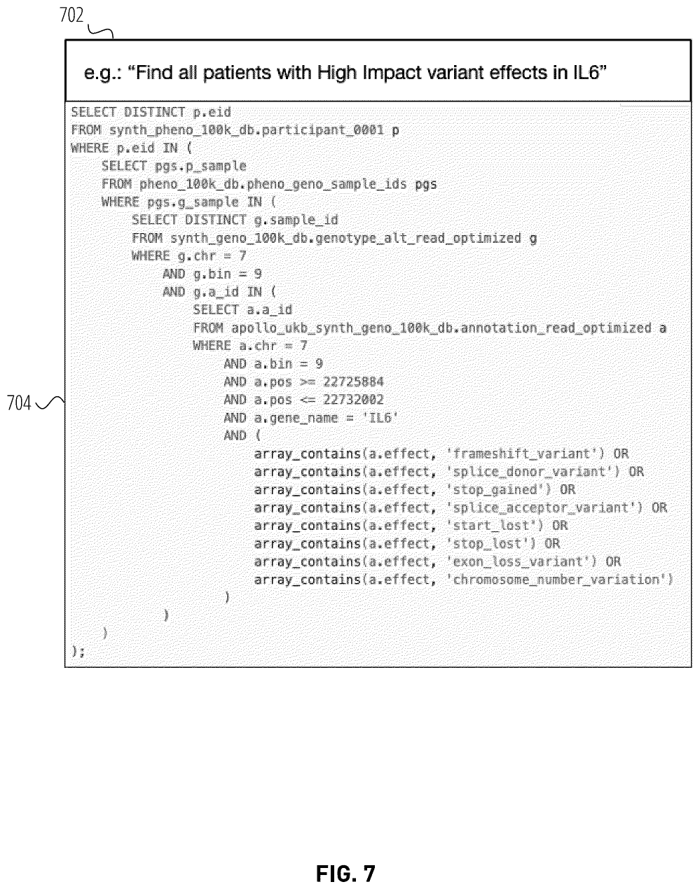

Moreover, the subject technology provides improvements over other platforms by automatically interpreting natural language queries (e.g., “Find all patients with High Impact variant effects in IL6”) and converting such natural language queries into complex SQL queries (e.g., as discussed in FIG. 7) without user intervention. This eliminates the technical barriers present in other systems while maintaining the underlying sophistication required for accurate scientific analysis.

Embodiments of the subject technology implement a platform that uses Large Language Models (LLMs) to facilitate the query and analysis of clinico-omic datasets within the platform. These clinico-omic datasets include two main types of information:
Clinical data: This includes any raw or derived results from clinical trials, medical records data, or phenotypic data from human or model organism research.
Omics data: This can include any molecular results usually studied in molecular biology, such as genomic, transcriptomic, proteomic, or metabolomic results.

In an embodiment, users input natural language queries through a graphical user interface (GUI), wherein the queries may be formulated as questions or statements describing desired data analysis objectives. The subject system processes these natural language inputs along with contextual data through a Large Language Model (LLM), but rather than generating direct responses, the LLM is configured to create a structured analytical plan.

The subject system, for example, prompts the LLM with instructions to make a plan of steps such as: “You are working on dataset < . . . >. The user is asking < . . . >. You have at your disposal these tools: < . . . >. Please make a step-by-step plan.” This approach generates a systematic methodology comprising discrete, structured steps for addressing the user's query.

Each step in the generated plan may require iterative processing through recursive LLM interactions to achieve completion. For example, to implement a particular step, the subject system may generate a subsequent prompt to the LLM stating: “You are working on dataset < . . . >. You need to select which fields are related to < . . . >. Write out those fields.” This recursive questioning mechanism enables the subject system to break down complex analytical tasks into manageable tasks while maintaining contextual awareness throughout the process.

The structured planning approach allows the system to systematically address multi-faceted queries involving clinico-omic data analysis, ensuring that each component of a user's request is properly interpreted and executed through appropriate tool selection and data field identification.

When the LLM generates an incorrect or unsatisfactory response during step execution, the subject system employs corrective mechanisms to improve subsequent performance. The system may implement prompt augmentation by providing additional contextual examples to guide the LLM toward correct responses. For instance, the system may enhance the prompt with exemplary guidance such as: “For example, if we were asking you for fields related to gender, you would have given us field ABC.”

Alternatively, the subject system may employ fine-tuning methodologies to retrain the LLM's parameters for improved accuracy. Fine-tuning involves providing the LLM with specific question-answer pairs and the desired reasoning and tool use trace leading to the correct answer, that demonstrate the desired response pattern. For example, the system may present a training pair where Q=“You are working on dataset < . . . >. You need to select which fields are related to <gender>. Write out those fields”, A=“The field is ABC,” then adjust the LLM's weights through retraining to enable generation of the correct answer.

This dual approach of prompt enhancement and fine-tuning enables the system to continuously improve its performance in field identification and query interpretation tasks. The corrective mechanisms ensure that the LLM learns from previous errors and develops more accurate responses for similar queries involving dataset field selection and mapping. These adaptive learning capabilities represent a significant advancement over static systems that cannot improve their performance based on at least error correction.

The platform may include functionality for a clinico-omics data assistant. For example, LLM's may be used to empower researchers who are not proficient in programming languages to query their clinico-omic data in a conversational, prompt-based interface. This enables researchers to ask questions of their data to find patients of interest (e.g., cohorts) using their typical scientific or clinical vernacular such as: “Find patients with renal cancer with tumor sequencing and gene expression data” or “Find patients with diabetes over 60 years old.”

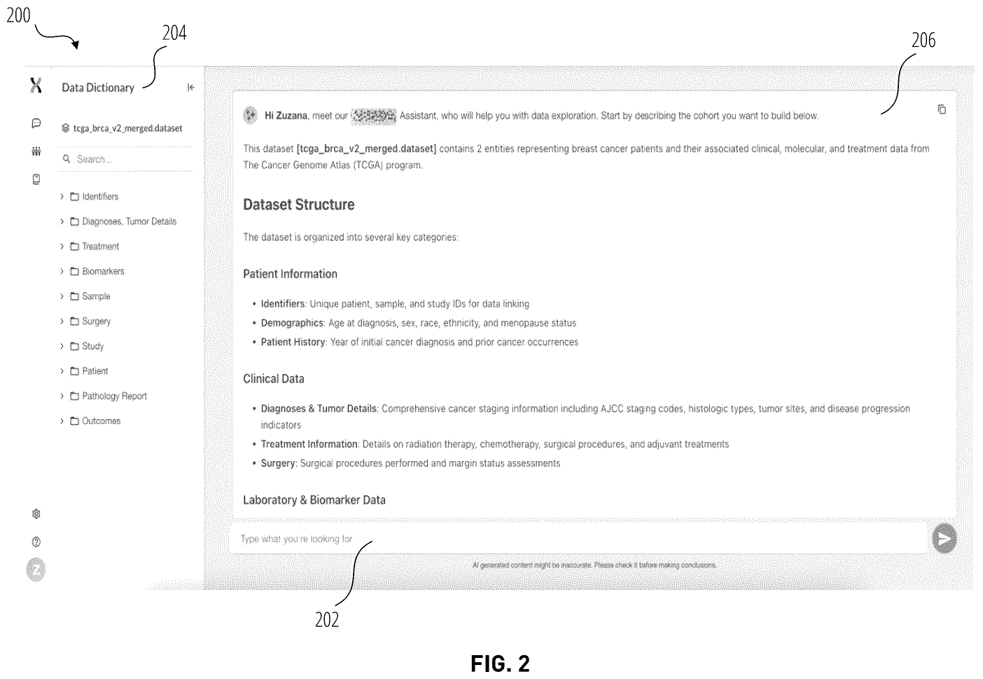

Since there is often judgement required to best match the user's question to the database content, a prompt-based interface allows the LLM to return to the user for clarification when multiple fields or values could be interpreted. Examples of this functionality is shown in FIGS. 2-5.

In an example, the LLM may prompt will also support longitudinal questions of increasing complexity, such as: Find patients who had a change in HR (ER or PR) or HER2 status after a metastatic event. Find patients that had an increase (or decrease) in ccog_score or karnofsky_score after first administration of palbociclib.

The subject platform also provides intelligent analysis that moves beyond location of patients of interest into cohorts and allows non-technical users to perform basic statistical functions on the query results returned.

The platform may provide a cloud infrastructure, e.g., using Amazon Web Services (AWS), and the like. This may provide on-demand, scalable computing and storage resources for users of the platform. In some examples, the platform provides a variety of bioinformatics tools. Access to a library of pre-built bioinformatics tools and apps for genomic analysis is provided in some examples. Users can also integrate their own custom tools with the platform. Further, in some examples, workflow languages are provided. For example, multiple workflow languages, including Nextflow and WDL (Workflow Description Language), may enable users to create and run complex bioinformatics pipelines. For example, the platform may allow users to integrate their own tools using Docker containers, providing flexibility and reproducibility. The platform may provide JupyterLab functionality where JupyterLab notebooks are used for data analysis and visualization.

In some examples, the platform is configured to provide robust APIs to enable integrations with existing systems and automation of workflows. The platform data management functionality is provided in some examples where tools for managing large-scale genomic and clinical data, including metadata tagging and search capabilities. Further, security and compliance may be provided. To this end, platform incorporates various security measures and compliance standards (e.g., HIPAA, ISO27001, GxP) to ensure data protection and regulatory adherence. The platform may also include collaboration tools for secure data sharing and collaboration among distributed teams.

The platform may deploy Artificial Intelligence (AI) and Machine Learning (ML) to provide AI and ML algorithms for advanced analytics. By combining two or more of the technologies described above, a comprehensive ecosystem platform may be provided for managing, analyzing, and collaborating on precision health data, particularly in the realm of genomics and multiomics research. In an example, the LLM is trained to understand the scientific vernacular and convert that behind the scenes to what the system needs to query (e.g. SQL against a specific database). This can be done for either existing data models so as to train up-front, or for new data models (e.g., customer-specific data models). The format by the LLM may be either a prompt text, or when the query has been resolved adequately, the query may be sent to a back-end via a data access layer.

Computing resources used by one or more machines, databases, or networks may be more efficiently utilized or even reduced. Examples of such computing resources can include processor cycles, network traffic, memory usage, graphics processing unit (GPU) resources, data storage capacity, power consumption, or cooling capacity.

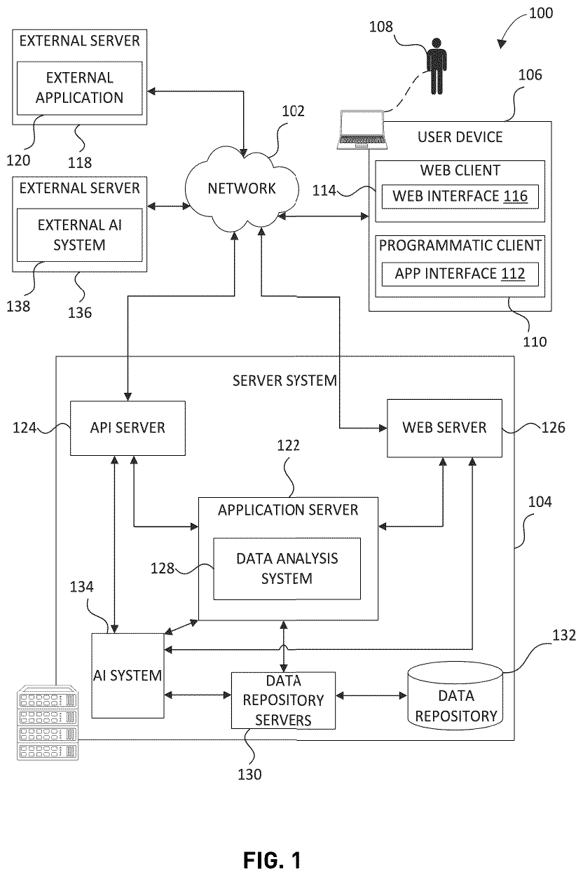

FIG. 1 is a diagrammatic representation of a networked computing environment 100 in which some examples of the present disclosure may be implemented or deployed. One or more servers in a server system 104 provide server-side functionality via a network 102 to a networked device, in the example form of a user device 106 that is accessed by a user 108. A web client 114 (e.g., a browser) or a programmatic client 110 (e.g., an “app”) may be hosted and executed on the user device 106. The server system 104 may include components from a backend architecture 600 or application architecture 800 as discussed further herein. The programmatic client 110 may include components from the application architecture 800 as discussed further herein.

An Application Program Interface (API) server 124 and a web server 126 provide respective programmatic and web interfaces to components of the server system 104. A specific application server 122 hosts a data analysis system 128 (e.g., a biomedical data analysis system), which includes components, modules, or applications.

The user device 106 can communicate with the application server 122, such as via the web interface supported by the web server 126 or via the programmatic interface provided by the API server 124. It will be appreciated that, although only a single user device 106 is shown in FIG. 2, a plurality of user devices may be communicatively coupled to the server system 104 in some examples. Further, while certain functions may be described herein as being performed at either the user device 106 (e.g., web client 114 or programmatic client 110) or the server system 104, the location of certain functionality either within the user device 106 or the server system 104 may be a design choice.

The application server 122 is communicatively coupled to one or more data repository servers 130, facilitating access to a data repository 132 (e.g., a database) or multiple data repositories. In some examples, the data repository 132 includes storage devices that store information to be processed by the data analysis system 128, such as biomedical data.

The application server 122 accesses application data to provide one or more applications or software tools to the user device 106 via a web interface 116 or an app interface 112. As described further below, the application server 122, using the data analysis system 128, may provide one or more tools or functions for biomedical diagnostics.

In some examples, the data analysis system 128 operates together with an AI system 134 of the server system 104. The AI system 134 can provide machine learning models and related functionality used for enhanced biomedical data analysis. The AI system 134 can provide various capabilities, such as training models, providing or obtaining predictions, and monitoring performance. The AI system 134 may leverage training datasets (e.g., stored in the data repository 132) to construct machine learning pipelines and train or re-train (e.g., adjust) machine learning models used by the data analysis system 128. In some examples, the AI system 134 provides a variety of services to different subsystems within the server system 104.

The AI system 134 may house or provide access to a generative machine learning model related processing capabilities. Generative AI is a term that may refer to any type of AI that can create new content. For example, generative machine learning model can produce text, images, video, audio, code, or synthetic data. In some examples, the generated content may be similar to the original data.

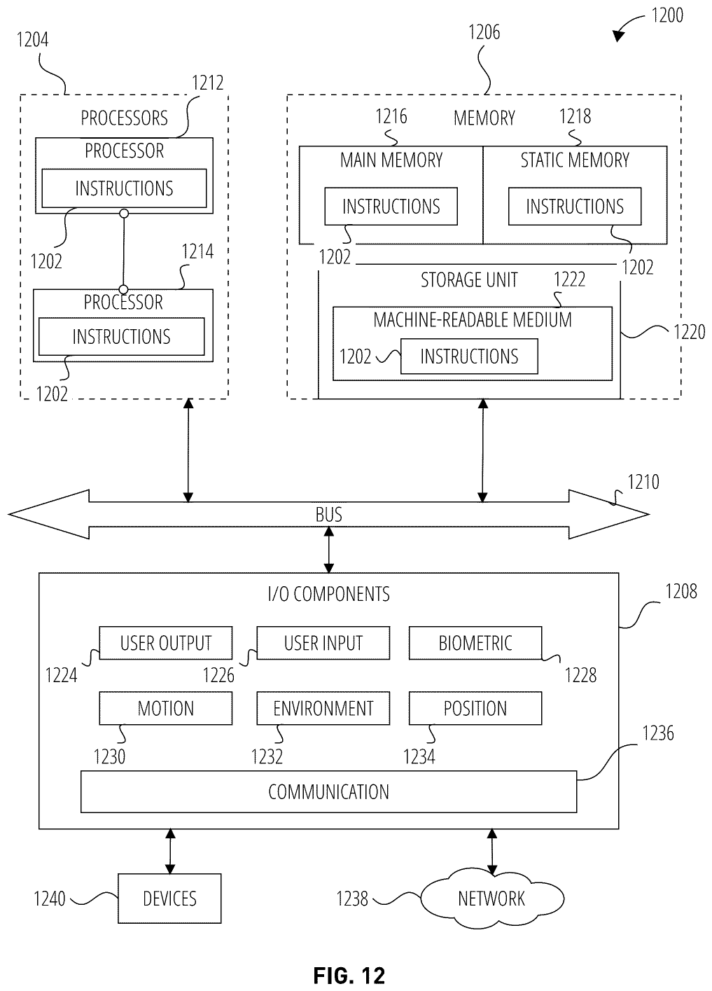

In some examples, the application server 122 is part of a cloud-based platform provided by a software service provider and that allows the user 108 to utilize tools or features of the data analysis system 128 and, optionally, other tools provided by the software service provider. For example, the user 108 is associated with a user account that has access to one or more of these tools or features. At least part of the application server 122, the data repository servers 130, the API server 124, the web server 126, and the data analysis system 128 may be implemented in a computer system, in whole or in part, as described below with respect to FIG. 12.

In some examples, external applications, such as an external application 120 executing on an external server 118, can communicate with the application server 122 via the programmatic interface provided by the API server 124. For example, a third-party application may support one or more features or functions on a website or platform hosted by a third party, or may perform certain methodologies and provide input or output information to the application server 122 for further processing or publication. Similarly, the AI system 134 may communicate with an external server 118 that hosts an external AI system 138 to benefit from features or functions of the external AI system 138. Accordingly, in some examples, at least some of the features or functions of the AI system 134 are provided or supported by the external AI system 138.

The network 102 may be any network (or multiple networks) that enables communication between or among machines, databases, and devices. Accordingly, the network 102 may be a wired network, a wireless network (e.g., a mobile or cellular network), or any suitable combination thereof. The network 102 may include one or more portions that constitute a private network, a public network (e.g., the Internet), or any suitable combination thereof.

Referring more broadly to the networked computing environment 100, the server system 104 may thus embody multiple subsystems, which are supported on the client-side (e.g., by the web client 114 or the programmatic client 110) and on the server-side (e.g., by one or more subsystems as described herein). In some examples, one or more of these subsystems are implemented as microservices. A microservice subsystem (e.g., a microservice application) may have components that enable it to operate independently and communicate with other services. Example components of a microservice subsystem may include:
Function logic: The function logic implements the functionality of the microservice subsystem, representing a specific capability or function that the microservice provides.
API interface: Microservices may communicate with other components through well-defined APIs or interfaces, using lightweight protocols such as representational state transfer (REST) or messaging. The API interface defines the inputs and outputs of the microservice subsystem and how it interacts with other microservice subsystems.
Data storage: A microservice subsystem may be responsible for its own data storage, which may be in the form of a database, cache, or other storage mechanism (e.g., using the data repository 132). This enables a microservice subsystem to operate independently of other microservices.
Service discovery: Microservice subsystems may find and communicate with other microservice subsystems. Service discovery mechanisms enable microservice subsystems to locate and communicate with other microservice subsystems in a scalable and efficient way.
Monitoring and logging: Microservice subsystems may need to be monitored and logged in order to ensure availability and performance. Monitoring and logging mechanisms enable the tracking of health and performance of a microservice subsystem.

### Example Use Cases:

Data element discovery—Does the dataset include ethnicity information?
Data filtering/cohort building—Filter for type 2 diabetics with pathogenic GCK variants.
Longitudinal (time axis) querying—Find those who were admitted for myocardial infarction and then within a month were diagnosed with GERD.
Compute simple insights (via SQL-like formulas)—What is the average BMI in that cohort?
Compute complex insights (via python code)—Compute Fisher's Exact Test between loss of function in the CFTR gene and diagnosis of cystic fibrosis
Create custom visualizations—Plot BRCA1 expression by gender and ethnicity.

FIG. 2 illustrates a user interface 200 for an omics data assistant that enables exploring clinico-omics datasets, in accordance with an embodiment of the subject technology. The clinico-omics data assistant may be referred to as the “AI assistant” or simply the “assistant” as mentioned elsewhere in this disclosure.

In the example of FIG. 2, the user interface 200 shows information indicating a particular clinico-omics dataset that will be queried against, corresponding to a breast cancer dataset (“tcga_brca_v2_merged.dataset”) that includes a number of entities representing breast cancer patients and their associated clinical, molecular, and treatment data from a particular program (e.g., “The Cancer Genome Atlas (TCGA) program”). It should be appreciated that any appropriate clinico-omics dataset can be utilized, and still be within the scope of the subject technology.

The user interface 200 is designed to facilitate data exploration through an organized hierarchical structure with interactive elements, and includes text input field 202 for receiving textual input for a natural language query. In an example, a natural language query is a request for information that is phrased in the way a person would speak or write, rather than using a formal, structured query language such as SQL. A given natural language query can be in the form of a question or a statement, and unlike a database query that requires specific keywords, commands, and punctuation (e.g., SELECT * FROM patients WHERE diagnosis=‘cancer’), a natural language query is free-form where a user does not need to know the underlying database structure or coding language.

The subject system enhances clinical data by combining SQL databases with custom data dictionaries to form integrated datasets. In an implementation, the data dictionary serves as a comprehensive metadata repository that provides structured information about database fields that would otherwise be cryptic to the user.

The user interface 200 includes a data dictionary panel 204. The data dictionary panel 204 displays a collapsible hierarchical data dictionary with expandable categories including:
Identifiers
Diagnoses, Tumor Details
Treatment
Biomarkers
Sample
Surgery
Study
Patient
Pathology Report
Outcomes

Each category is represented with expandable arrow indicators (>) that enables the user to drill down, via selection, into subcategories and specific data fields. This organization with categories allows the user to navigate complex biomedical datasets without requiring technical database knowledge. Further, by utilizing the data dictionary, cryptic database schemas can be represented as accessible, searchable metadata that facilitates the user to understand and interact with complex clinico-omics datasets more effectively.

The user interface 200 provides an interface area 206 that displays contextual information for data exploration. As shown, the interface area 206 includes a greeting message stating “Hi UserXYZ, meet our Assistant, who will help you with data exploration. Start by describing the cohort you want to build below.”

The interface area 206 also displays dataset information. As shown, interface area 206 provides comprehensive dataset structure information organized into key categories:

### 1) Patient Information Section Displaying:

Identifiers for unique patient, sample, and study IDs for data linking
Demographics including age at diagnosis, sex, race, ethnicity, and menopause status
Patient History covering year of initial cancer diagnosis and prior cancer occurrences

### 2) Clinical Data Section Showing:

Diagnoses & Tumor Details with comprehensive cancer staging information including AJCC staging codes, histologic types, tumor sites, and disease progression indicators
Treatment Information detailing radiation therapy, chemotherapy, surgical procedures, and adjuvant treatments
Surgery information covering surgical procedures performed and margin status assessments

### 3) Laboratory & Biomarker Data Section

To provide search functionality, the interface includes the text input field 202 with placeholder text “Type what you're looking for” enabling the user to query the dataset using input in the form of a natural language query.

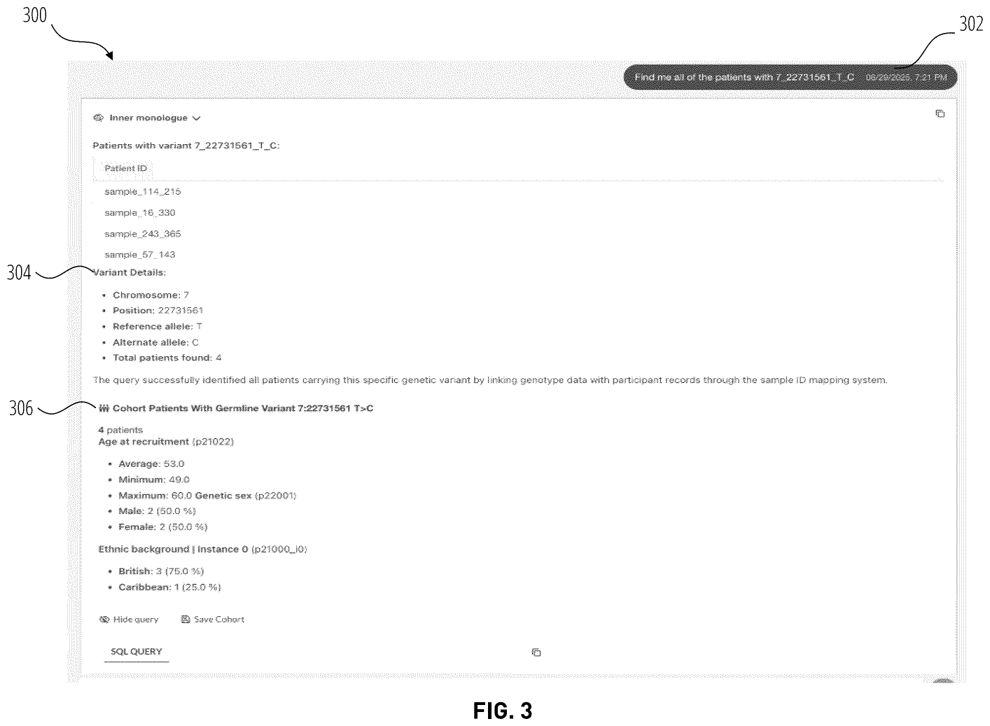

FIG. 3 illustrates a user interface 300 for the omics data assistant, in accordance with an embodiment of the subject technology. The user interface 300 displays information related to a cohort, in which the cohort represents a filtered subset of patients that results from processing a natural language query in the subject system.

In the example of FIG. 3, the user interface 300 shows a query-response model for genetic variant searches. For example, user interface 300 shows a natural language query 302 for a genetic variant search for “7_22731561_T_C” with results displaying four matching patients (e.g., patient IDs corresponding to sample_114_215, sample_16_330, sample_243_365, sample_57_143). Referring to FIG. 2, the natural language query 302 may have been provided as input in text input field 202. When the user inputs the natural language query 302, the subject system processes the natural language query 302 and generates a cohort as the primary result. The cohort represents the specific group of patients that match the criteria specified in the natural language query.

The user interface 300 displays information including variant details 304 including chromosome 7, position 22731561, reference allele T, and alternate allele C. Further information is provided in the user interface 300 that includes a message indicating that “[t]he query successfully identified all patients carrying this specific genetic variant by linking genotype data with participant records through the sample ID mapping system.”

The user interface 300 further displays cohort statistics 306 in a structured format showing the following information related to the cohort determined from successfully processing the natural language query 302:
Age demographics with average (53.0), minimum (49.0), and maximum (60.0) values
Genetic sex distribution (50% male, 50% female)
Ethnic background breakdown (75% British, 25% Caribbean)

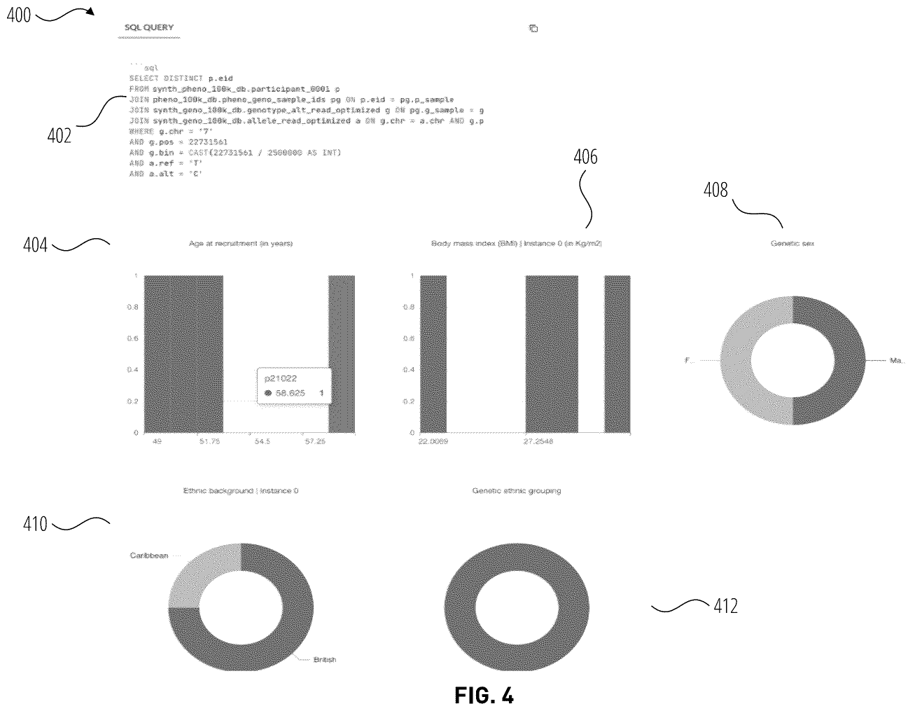

FIG. 4 illustrates an example of a user interface 400, in accordance with an embodiment of the subject technology. As shown, the user interface 400 may include different displays of additional information in connection with the natural language query 302 discussed in FIG. 3.

The user interface 400 prominently displays the underlying SQL query, represented by a SQL query 402, used to retrieve the genetic variant data. In the example of FIG. 4, the SQL query 402 shows a complex multi-table join operation that:
Selects distinct patient IDs (p.eid) from the participant table
Joins participant data with phenotype-genotype sample mapping tables
Filters for specific chromosome (7), position (22731561), and allele information (reference “T”, alternate “C”)
Uses binning optimization for efficient data retrieval

The user interface 400 also includes various statistical visualization charts. As shown, an age distribution chart 404 shows a histogram showing the age distribution of patients in the cohort, with the x-axis displaying age ranges and y-axis showing frequency distribution. A body mass index chart 406 shows a visualization displaying BMI distribution data for the identified patient cohort. A genetic sex distribution chart 408 includes a chart showing the gender breakdown of the cohort, displaying the 50% male and 50% female distribution mentioned in the patient statistics. An ethnic background chart 410 shows a chart representing the ethnic composition of the cohort, corresponding to the 75% British and 25% Caribbean breakdown shown in the statistical summary. A genetic ethnic grouping chart 412 includes additional ethnicity-related visualization that provides more granular ethnic classification data for the patient cohort.

These displays of various visualizations, in the user interface 400, demonstrates an ability to transform complex SQL query results into accessible, multi-dimensional data representations suitable for clinical and research analysis.

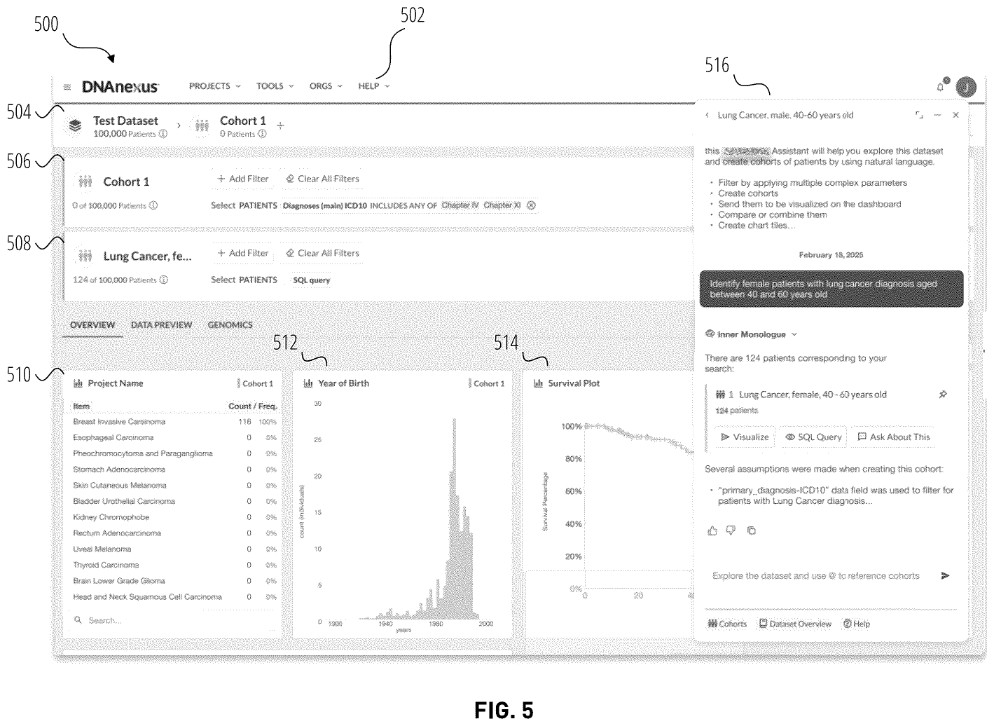

FIG. 5 illustrates an example of a user interface 500 that includes a combination of various graphical elements, in accordance with an embodiment of the subject technology.

In the example of FIG. 5, a menu bar 502 includes different menu items with expandable sections including “Projects,” “Tools,” “Orgs,” and “Help,” which provides the user with organized access to different system functionalities.

A dataset information panel 504 display information related to a given dataset for applying a natural language query, which determines a cohort.

The user interface 500 includes a cohort panel 506 showing “Cohort 1” with filtering capabilities. This panel includes options to “Add Filter” and “Clear All Filters” with a patient count indicator showing “0 of 100,000 Patients” that updates based on applied filters. The panel also displays the current filter criteria, showing “Diagnoses (main) ICD10 INCLUDES ANY OF Chapter IV Chapter XI” indicating the active querying parameters for the SQL query (e.g., Select PATIENTS).

The user interface 500 also includes lung cancer cohort panel 508 showing “Lung Cancer, fc . . . ” with filtering capabilities. This panel includes options to “Add Filter” and “Clear All Filters” with a patient count indicator showing “124 of 100,000 Patients” that updates dynamically based on applied filters.

The user interface 500 includes three additional graphical areas. A project name distribution chart 510 includes a bar chart showing the distribution of patients across different project names, with “Breast Invasive Carcinoma” showing 116 patients (100%), and other cancer types showing 0 patients, including Esophageal Carcinoma, Pheochromocytoma and Paraganglioma, Stomach Adenocarcinoma, and others. A year of birth distribution chart 512 shows a histogram that displays the year of birth distribution for the cohort, showing patient counts across different birth years from approximately 1940 to 2000, with the y-axis indicating patient frequency and the x-axis showing calendar years. A survival plot chart 514 showing survival percentage over time, with the y-axis displaying survival percentage from 0% to 100% and the x-axis showing time progression. The plot demonstrates the system's capability to generate sophisticated clinical outcome visualizations.

The aforementioned interface elements collectively demonstrate an ability to provide comprehensive cohort management, dynamic filtering, and automated generation of clinically relevant visualizations for biomedical research applications.

The user interface 500 further includes a floating panel 516 that displays information for the lung cancer cohort, specifically showing “Lung Cancer, male, 40-60 years old” with associated patient counts and management options. This panel includes functionality for visualization, SQL query review, and additional analysis options.

The floating panel 516 includes a greeting message and input field. The floating panel 516 shows “<Lung Cancer, male, 40-60 years old” at the top, indicating the current context.

The floating panel 516 includes information indicating a natural language query, with example text showing “Identify female patients with lung cancer diagnosis aged between 40 and 60 years old.” The floating panel 516 displays the AI assistant's response, including “There are 124 patients corresponding to your search” and shows a new cohort that was created and labeled “Lung Cancer, female, 40-60 years old, 124 patients.” The floating panel 516 includes selectable buttons and options such as “Visualize,” “SQL Query,” and “Ask About This,” allowing users to further explore the generated results. Further, floating panel 516 provides contextual information about the search process, including a section titled “Several assumptions were made when creating this cohort” with explanatory text about how the system interpreted the user's query, specifically noting that “primary_diagnosis-ICD10” data field was used to filter for patients with Lung Cancer diagnosis.

Also shown, the floating panel 516 incudes a text input field (e.g., “Explore the dataset and use @ to reference cohorts”) for entering a natural language query, and additional interface features, such as options for “Cohorts,” “Dataset Overview,” and “Help.”

This floating panel design represents an advancement over other database interfaces by providing a conversational, context-aware interface that guides the user through complex data queries while maintaining transparency about the underlying analytical processes.

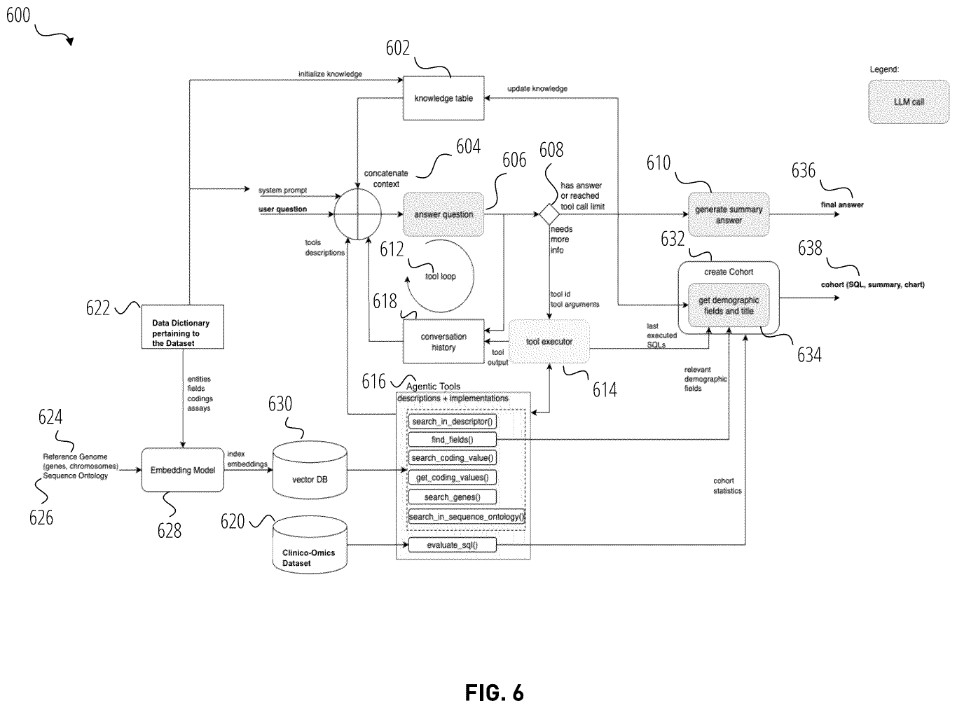

FIG. 6 illustrates an example of a backend architecture 600, in accordance with an embodiment of the subject technology.

In an example, the backend architecture 600 includes various tools that are implemented as functions that a LLM knows how to call, designed to extend LLM intrinsic functionality and knowledge while connecting to external data sources. The backend architecture 600, in an implementation, provides two types of tools:
1. Specialized semantic search tools: These include search capabilities in clinico-omics dataset descriptor, fields, codings, genes, and sequence ontology
2. SQL evaluation tools: These evaluate SQL queries against databases in a clinico-omics dataset

The backend architecture 600 includes a Large Language Model (LLM) ReAct agent that implements a reasoning and acting framework with tool calls, which processes natural language queries and generates structured responses for clinico-omics data analysis. The backend architecture 600 performs an iterative processing loop that combines reasoning capabilities with external tool execution to provide comprehensive data analysis results.

The backend architecture 600 includes a knowledge table 602 that serves as an episodic memory component, maintaining persistent information across multiple user interactions. In an example, two knowledge operations are implemented:
1. Initialize Knowledge: Establishes baseline knowledge parameters at system startup
2. Update Knowledge: Continuously incorporates new information learned during tool execution cycles into the knowledge table for future reference

The backend architecture 600 implements a context concatenation function 604 that combines multiple information sources to create comprehensive input for the LLM. This function can utilize the following:
1. A system prompt including behavioral instructions and tool definitions. In an implementation, the system prompt includes instructions that define the LLM behavior and includes general information about the “Data Dictionary pertaining to the Dataset,” including primary key information.
2. User question(s) (e.g., corresponding to natural language queries)
3. A conversation history 618 from previous user interactions and tool execution(s)
4. Knowledge table contents with prioritized episodic memory
5. Tool descriptions and available functionality specifications

An “answer question” LLM call 606 is performed using at least the aforementioned information related to the concatenated context. As part of a decision-making and control flow, the LLM processes the concatenated context and makes decisions through a binary evaluation 608 that determines whether the LLM “has answer or reached tool call limit” or “needs more info.” This decision point controls the overall processing flow and determines whether to proceed with a “generate summary answer” LLM call 610 or continue with additional tool execution.

When the backend architecture 600 determines that more information is needed, it enters a tool loop that performs the following operations:
1. Tool Selection: The backend architecture 600 identifies appropriate tools based on the user question and available tool descriptions.
2. Argument Generation: The backend architecture 600 generates specific tool information (e.g., tool id and tool arguments) required for tool execution.
3. Tool Execution: A tool executor 614 selects a tool from a set of agentic tools 616 and executes the selected tool using the tool information, and provides a tool output.
4. Tool output Processing: The backend architecture 600 receives and incorporates the tool output into the conversation history 618.
5. Context concatenation function 604 Execution: The backend architecture 600 executes the context concatenation function 604 using at least the updated conversation history 618.
6. “Answer question” LLM call 606 Execution: the backend architecture 600 performs the “answer question” LLM call 606 using at least the updated output from the context concatenation function 604
7. Binary evaluation 608 Execution: the backend architecture 600 again performs the binary evaluation 608 to determine whether more information is needed or whether the “generate summary answer” LLM call 610 can be performed.

As mentioned above, the backend architecture 600 includes the set of agentic tools 616, which includes multiple specialized tools designed for clinico-omics data analysis, which may include the following functions:
search_in_descriptor( ): Searches dataset metadata and descriptive information
find_fields( ): Identifies relevant data fields within the dataset structure
search_coding_value( ): Searches medical coding systems and value mappings
get_coding_values( ): Retrieves specific coding values and their meanings
search_genes( ): Searches genomic information and gene-related data
search_in_sequence_ontology( ): Searches biological sequence ontology databases
evaluate_sql( ): Executes and validates SQL queries against a clinico-omics dataset 620

The backend architecture 600 integrates multiple external data sources to enhance its analytical capabilities, which can include the following:
A data dictionary 622 that includes dataset-specific metadata and field descriptions
A reference genome 624 that includes genomic reference information including genes and chromosomes
A sequence ontology 626 that includes information related to biological terminology and classification systems

The backend architecture 600 uses an embedding model 628 to vectorize the external data sources such as 622 and 624 and stores the embedding vectors together with the textual representation of the data in a vector database 630 for later use by the tools specialized in semantic-search from the set of Agentic tools 613.

The backend architecture 600, upon reaching a satisfactory answer or tool call limit, proceeds through final processing operations, including the following:
1. The “generate summary answer” LLM call 610 execution: The LLM formulates a comprehensive response based on accumulated information and creates a structured summary of findings and analysis results as a final answer output 636.
2. A create cohort operation 632 that performs a “get demographic fields and title” LLM call 634 and generates patient cohort definitions with associated SQL queries, statistical summaries, and visualization charts as a cohort output 638. As shown, the create cohort operation 632 or the “get demographic fields and title” LLM call 634 can utilize information related to last executed SQLs, relevant demographic fields, and cohort statistics are part of generating the cohort output 638.

This backend architecture 600 is enabled to process complex natural language queries about clinico-omics data and generate sophisticated analytical results while maintaining contextual awareness and leveraging specialized domain knowledge throughout the processing workflow.

FIG. 7 illustrates examples of a user question 702 and a complex query 704 generated by the omics data assistant, in accordance with an embodiment of the subject technology.

The user question 702 corresponds to a complex natural language query e.g., “Find all patients with High Impact variant effects in IL6,” which the clinico-omics data assistant converts into a SQL query corresponding to the complex query 704, including multiple table joins, genomic coordinate filtering, and variant effect analysis. In an example, complex SQL when they include multiple layer of logic. In an example, a complex query can refer to a query that includes multiple layers of logic.

The clinico-omics data assistant therefore can process arbitrary natural language text prompts as input and responds with markdown-formatted free text combined with structured data including cohort definitions, SQL queries, statistics, and charts.

The clinico-omics data assistant demonstrates comprehensive understanding of the structure of clinico-omics datasets and genomic terminology, including knowledge of sequence ontology and specialized prompts for biological data analysis. This enables the assistant to generate cohorts and create complex SQL queries for both phenotypic questions and genomic questions.

The clinico-omics data assistant operates through sophisticated internal processes that include:
Autonomous Reasoning: The clinico-omics data assistant reasons about what information is necessary to generate correct answers.
Tool Call Management: The clinico-omics data assistant autonomously calls external functions (tools) and generates appropriate arguments for those functions.
Information Processing: The clinico-omics data assistant can find specific required information within potentially large tool outputs.
Decision Making: The clinico-omics data assistant determines whether additional tool calls are needed or if a satisfactory answer has been obtained.

In an example, a satisfactory answer is obtained when:
Sufficient Information Gathering: The subject system has collected enough relevant information through tool execution to address the user's natural language query. This would include successful identification of relevant data fields, appropriate coding values, and necessary dataset metadata.
Successful Query Generation: The subject system can generate valid SQL queries that properly address the user's request. The evaluate_sql( ) tool plays a critical role in determining whether generated queries execute successfully against the clinico-omics dataset.
Cohort Definition Completion: The subject system can create meaningful patient cohort definitions with associated statistical summaries and visualization charts that respond to the user's original query.

In an example, the clinico-omics data assistant also includes advanced error handling capabilities, demonstrating the ability to understand error messages from tools and perform auto-correction without user intervention.

In an implementation, the clinico-omics data assistant maintains an internal knowledge table that serves as episodic memory, updating this knowledge base with prioritized information learned during interactions. This allows the system to build upon previous interactions and maintain contextual awareness across multiple user sessions.

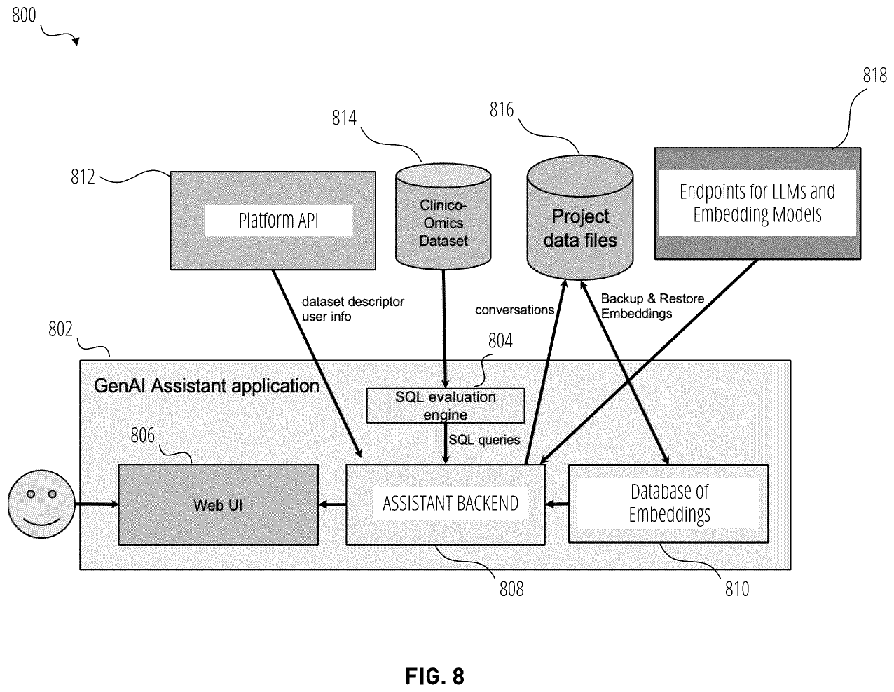

FIG. 8 illustrates an example of an application architecture 800 architecture, in accordance with an embodiment of the subject technology.

The application architecture 800 includes a GenAI assistant application 802 that serves as a primary processing engine that orchestrates system operations. The GenAI assistant application 802 integrates with multiple external components and manages the overall workflow for natural language query processing and data analysis.

The GenAI assistant application 802 includes a SQL evaluation engine 804 that executes generated SQL queries against the clinico-omics datasets. This SQL evaluation engine 804 validates query syntax, processes database operations, and returns results for further analysis.

The GenAI assistant application 802 includes a web UI 806 that provides the user interface(s) for natural language interactions, while an assistant backend 808 handles the server-side processing and coordination between different system components.

The GenAI assistant application 802 integrates with a platform API 812 to access platform services and manage data operations. This integration enables the GenAI assistant application 802 to interact with the platform ecosystem and leverage existing platform capabilities.

The GenAI assistant application 802 directly interfaces with a clinico-omics dataset 814, which includes biomedical data including clinical phenotypic information and molecular omics data. The GenAI assistant application 802 processes queries against these datasets to generate patient cohorts and analytical results.

The GenAI assistant application 802 accesses project and data files 816 that include dataset descriptors and user information. Such files can provide metadata and configuration information that enables the GenAI assistant application 802 to understand dataset structures and user contexts.

The GenAI assistant application 802, via the assistant backend 808, accesses a database of embeddings 810 that stores vectorized representations of dataset metadata, enabling efficient semantic searches and query matching. This database of embeddings 810 supports the ability to map natural language terms to appropriate database fields and concepts.

The GenAI assistant application 802 connects to endpoints for LLMs and embedding models 818, providing the core natural language processing capabilities. Such endpoints enable the GenAI assistant application 802 to interpret user queries, generate responses, and coordinate tool execution. The GenAI assistant application 802 also uses embedding models that convert textual information into vector representations for semantic matching and search capabilities. Such embedding models enable the GenAI assistant application 802 to understand relationships between user queries and dataset metadata.

The application architecture 800 maintains persistent storage for user conversations, enabling the AI assistant to maintain context across multiple interactions and build upon previous exchanges. The application architecture 800 includes backup and restore functionality for the embeddings database, ensuring data persistence and system reliability for the vectorized metadata that supports semantic search capabilities.

This application architecture 800 therefore enables the GenAI assistant application 802 to process natural language queries, generate appropriate database operations, and provide intelligent responses while maintaining integration with the broader platform ecosystem and leveraging advanced AI capabilities for biomedical data analysis.

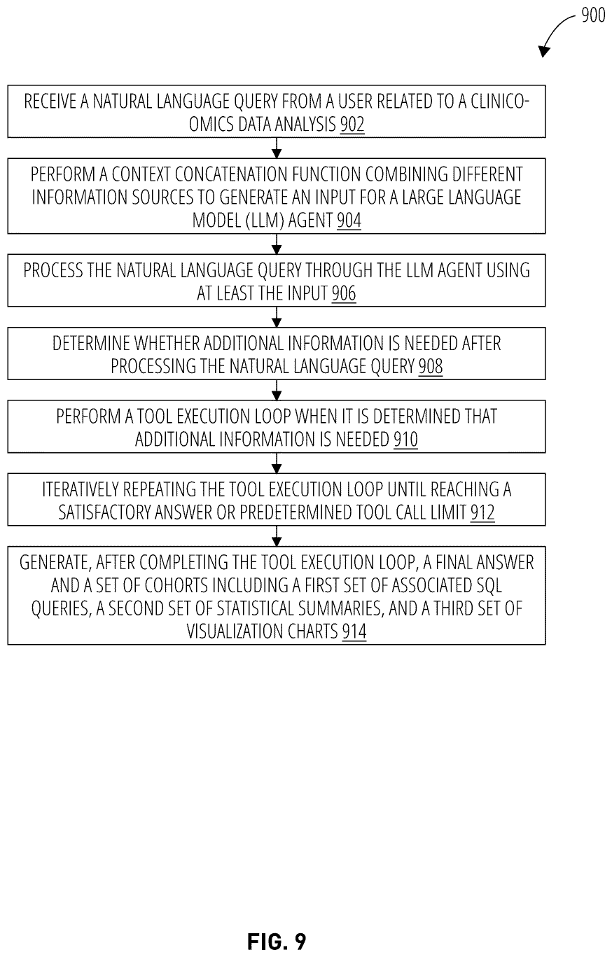

FIG. 9 is a flow diagram illustrating operations of a system (e.g., backend architecture 600) in performing a method 900, in accordance with some embodiments of the present disclosure. The method 900 may be embodied in computer-readable instructions for execution by one or more hardware components (e.g., one or more processors) such that the operations of the method 900 may be performed by components of the server system 104 or backend architecture 600. Accordingly, the method 900 is described below, by way of example with reference thereto. However, it shall be appreciated that method 900 may be deployed on various other hardware configurations and is not intended to be limited to deployment within the server system 104 or backend architecture 600.

In operation 902, backend architecture 600 receives a natural language query from a user related to a clinico-omics data analysis. In operation 904, backend architecture 600 performs a context concatenation function combining different information sources to generate an input for a large language model (LLM) agent. In operation 906, backend architecture 600 processes the natural language query through the LLM agent using at least the input. In operation 908, backend architecture 600 determines whether additional information is needed after processing the natural language query. In operation 910, backend architecture 600 performs a tool execution loop when it is determined that additional information is needed. In operation 912, backend architecture 600 iteratively repeating the tool execution loop until reaching a satisfactory answer or predetermined tool call limit (e.g., a predetermined maximum number of tool executions to prevent infinite loops, even if a fully satisfactory answer has not been achieved). In operation 914, backend architecture 600 generates, after completing the tool execution loop, a final answer and a set of cohorts including a first set of associated SQL queries, a second set of statistical summaries, and a third set of visualization charts.

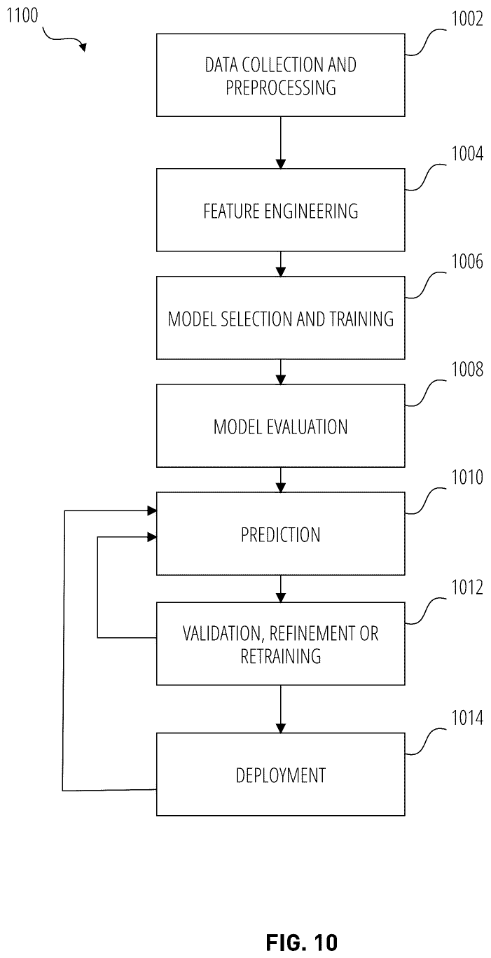

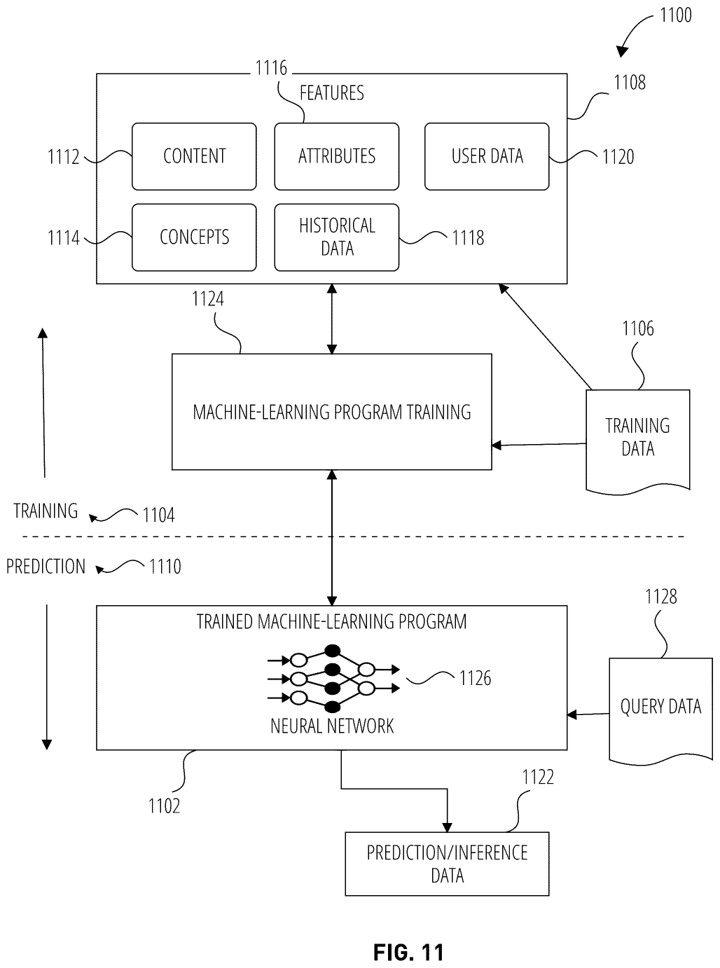

FIG. 10 is a flowchart depicting a machine-learning pipeline 1100, according to some examples. The machine-learning pipeline 1100 may be used to generate a trained model, for example the trained machine-learning program 1102 of FIG. 11, to perform operations associated with searches and query responses.

Broadly, machine learning may involve using computer algorithms to automatically learn patterns and relationships in data, potentially without the need for explicit programming. Machine learning algorithms can be divided into three main categories: supervised learning, unsupervised learning, and reinforcement learning.
Supervised learning involves training a model using labeled data to predict an output for new, unseen inputs. Examples of supervised learning algorithms include linear regression, decision trees, and neural networks.
Unsupervised learning involves training a model on unlabeled data to find hidden patterns and relationships in the data. Examples of unsupervised learning algorithms include clustering, principal component analysis, and generative models like autoencoders.
Reinforcement learning involves training a model to make decisions in a dynamic environment by receiving feedback in the form of rewards or penalties. Examples of reinforcement learning algorithms include Q-learning and policy gradient methods.

Examples of specific machine learning algorithms that may be deployed, according to some examples, include logistic regression, which is a type of supervised learning algorithm used for binary classification tasks. Logistic regression models the probability of a binary response variable based on one or more predictor variables. Another example type of machine learning algorithm is Naïve Bayes, which is another supervised learning algorithm used for classification tasks. Naïve Bayes is based on Bayes' theorem and assumes that the predictor variables are independent of each other. Random Forest is another type of supervised learning algorithm used for classification, regression, and other tasks. Random Forest builds a collection of decision trees and combines their outputs to make predictions. Further examples include neural networks, which consist of interconnected layers of nodes (or neurons) that process information and make predictions based on the input data. Matrix factorization is another type of machine learning algorithm used for recommender systems and other tasks. Matrix factorization decomposes a matrix into two or more matrices to uncover hidden patterns or relationships in the data. Support Vector Machines (SVM) are a type of supervised learning algorithm used for classification, regression, and other tasks. SVM finds a hyperplane that separates the different classes in the data. Other types of machine learning algorithms include decision trees, k-nearest neighbors, clustering algorithms, and deep learning algorithms such as convolutional neural networks (CNN), recurrent neural networks (RNN), and transformer models. The choice of algorithm depends on the nature of the data, the complexity of the problem, and the performance requirements of the application.

The performance of machine learning models is typically evaluated on a separate test set of data that was not used during training to ensure that the model can generalize to new, unseen data.

Although several specific examples of machine learning algorithms are discussed herein, the principles discussed herein can be applied to other machine learning algorithms as well. Deep learning algorithms such as convolutional neural networks, recurrent neural networks, and transformers, as well as more traditional machine learning algorithms like decision trees, random forests, and gradient boosting may be used in various machine learning applications.

Two example types of problems in machine learning are classification problems and regression problems. Classification problems, also referred to as categorization problems, aim at classifying items into one of several category values (for example, is this object an apple or an orange?). Regression algorithms aim at quantifying some items (for example, by providing a value that is a real number).

Generating a trained machine-learning program 1102 may include multiple phases that form part of the machine-learning pipeline 1100, including for example the following phases illustrated in FIG. 10:
Data collection and preprocessing 1002: This phase may include acquiring and cleaning data to ensure that it is suitable for use in the machine learning model. This phase may also include removing duplicates, handling missing values, and converting data into a suitable format.
Feature engineering 1004: This phase may include selecting and transforming the training data 1106 to create features that are useful for predicting the target variable. Feature engineering may include (1) receiving features 1108 (e.g., as structured or labeled data in supervised learning) and/or (2) identifying features 1108 (e.g., unstructured or unlabeled data for unsupervised learning) in training data 1106.
Model selection and training 1006: This phase may include selecting an appropriate machine learning algorithm and training it on the preprocessed data. This phase may further involve splitting the data into training and testing sets, using cross-validation to evaluate the model, and tuning hyperparameters to improve performance.
Model evaluation 1008: This phase may include evaluating the performance of a trained model (e.g., the trained machine-learning program 1102) on a separate testing dataset. This phase can help determine if the model is overfitting or underfitting and determine whether the model is suitable for deployment.
Prediction 1010: This phase involves using a trained model (e.g., trained machine-learning program 1102) to generate predictions on new, unseen data.
Validation, refinement or retraining 1012: This phase may include updating a model based on feedback generated from the prediction phase, such as new data or user feedback.
Deployment 1014: This phase may include integrating the trained model (e.g., the trained machine-learning program 1102) into a more extensive system or application, such as a web service, mobile app, or IoT device. This phase can involve setting up APIs, building a user interface, and ensuring that the model is scalable and can handle large volumes of data.

FIG. 11 illustrates further details of two example phases, namely a training phase 1104 (e.g., part of the model selection and trainings 1006) and a prediction phase 1110 (part of prediction 1010). Prior to the training phase 1104, feature engineering 1004 is used to identify features 1108. This may include identifying informative, discriminating, and independent features for effectively operating the trained machine-learning program 1102 in pattern recognition, classification, and regression. In some examples, the training data 1106 includes labeled data, known for pre-identified features 1108 and one or more outcomes. Each of the features 1108 may be a variable or attribute, such as an individual measurable property of a process, article, system, or phenomenon represented by a dataset (e.g., the training data 1106). Features 1108 may also be of different types, such as numeric features, strings, and graphs, and may include one or more of content 1112, concepts 1114, attributes 1116, historical data 1118, and/or user data 1120, merely for example.

In training phase 1104, the machine-learning pipeline 1100 uses the training data 1106 to find correlations among the features 1108 that affect a predicted outcome or prediction/inference data 1122.

With the training data 1106 and the identified features 1108, the trained machine-learning program 1102 is trained during the training phase 1104 during machine-learning program training 1124. The machine-learning program training 1124 appraises values of the features 1108 as they correlate to the training data 1106. The result of the training is the trained machine-learning program 1102 (e.g., a trained or learned model).

Further, the training phase 1104 may involve machine learning, in which the training data 1106 is structured (e.g., labeled during preprocessing operations). The trained machine-learning program 1102 implements a neural network 1126 capable of performing, for example, classification and clustering operations. In other examples, the training phase 1104 may involve deep learning, in which the training data 1106 is unstructured, and the trained machine-learning program 1102 implements a deep neural network 1126 that can perform both feature extraction and classification/clustering operations.

In some examples, a neural network 226 may be generated during the training phase 1104, and implemented within the trained machine-learning program 1102. The neural network 1126 includes a hierarchical (e.g., layered) organization of neurons, with each layer consisting of multiple neurons or nodes. Neurons in the input layer receive the input data, while neurons in the output layer produce the final output of the network. Between the input and output layers, there may be one or more hidden layers, each consisting of multiple neurons.

Each neuron in the neural network 1126 operationally computes a function, such as an activation function, which takes as input the weighted sum of the outputs of the neurons in the previous layer, as well as a bias term. The output of this function is then passed as input to the neurons in the next layer. If the output of the activation function exceeds a certain threshold, an output is communicated from that neuron (e.g., transmitting neuron) to a connected neuron (e.g., receiving neuron) in successive layers. The connections between neurons have associated weights, which define the influence of the input from a transmitting neuron to a receiving neuron. During the training phase, these weights are adjusted by the learning algorithm to optimize the performance of the network. Different types of neural networks may use different activation functions and learning algorithms, affecting their performance on different tasks. The layered organization of neurons and the use of activation functions and weights enable neural networks to model complex relationships between inputs and outputs, and to generalize to new inputs that were not seen during training.

In some examples, the neural network 1126 may also be one of several different types of neural networks, such as a single-layer feed-forward network, a Multilayer Perceptron (MLP), an Artificial Neural Network (ANN), a Recurrent Neural Network (RNN), a Long Short-Term Memory Network (LSTM), a Bidirectional Neural Network, a symmetrically connected neural network, a Deep Belief Network (DBN), a Convolutional Neural Network (CNN), a Generative Adversarial Network (GAN), an Autoencoder Neural Network (AE), a Restricted Boltzmann Machine (RBM), a Hopfield Network, a Self-Organizing Map (SOM), a Radial Basis Function Network (RBFN), a Spiking Neural Network (SNN), a Liquid State Machine (LSM), an Echo State Network (ESN), a Neural Turing Machine (NTM), or a Transformer Network, merely for example.

In addition to the training phase 1104, a validation phase may be performed on a separate dataset known as the validation dataset. The validation dataset is used to tune the hyperparameters of a model, such as the learning rate and the regularization parameter. The hyperparameters are adjusted to improve the model's performance on the validation dataset.

Once a model is fully trained and validated, in a testing phase, the model may be tested on a new dataset. The testing dataset is used to evaluate the model's performance and ensure that the model has not overfitted the training data.

In prediction phase 1110, the trained machine-learning program 1102 uses the features 1108 for analyzing query data 1128 to generate inferences, outcomes, or predictions, as examples of a prediction/inference data 1122. For example, during prediction phase 1110, the trained machine-learning program 1102 generates an output. Query data 1128 is provided as an input to the trained machine-learning program 1102, and the trained machine-learning program 1102 generates the prediction/inference data 1122 as output, responsive to receipt of the query data 1128.

In some examples, the trained machine-learning program 1102 may be a generative AI model. Generative AI is a term that may refer to any type of artificial intelligence that can create new content from training data 1106. For example, generative AI can produce text, images, video, audio, code, or synthetic data similar to the original data but not identical.

Some of the techniques that may be used in generative AI are:
Convolutional Neural Networks (CNNs): CNNs may be used for image recognition and computer vision tasks. CNNs may, for example, be designed to extract features from images by using filters or kernels that scan the input image and highlight important patterns.
Recurrent Neural Networks (RNNs): RNNs may be used for processing sequential data, such as speech, text, and time series data, for example. RNNs employ feedback loops that allow them to capture temporal dependencies and remember past inputs.
Generative adversarial networks (GANs): GNNs may include two neural networks: a generator and a discriminator. The generator network attempts to create realistic content that can “fool” the discriminator network, while the discriminator network attempts to distinguish between real and fake content. The generator and discriminator networks compete with each other and improve over time.
Variational autoencoders (VAEs): VAEs may encode input data into a latent space (e.g., a compressed representation) and then decode it back into output data. The latent space can be manipulated to generate new variations of the output data. VAEs may use self-attention mechanisms to process input data, allowing them to handle long text sequences and capture complex dependencies.
Transformer models: Transformer models may use attention mechanisms to learn the relationships between different parts of input data (such as words or pixels) and generate output data based on these relationships. Transformer models can handle sequential data, such as text or speech, as well as non-sequential data, such as images or code.

In generative AI examples, the output prediction/inference data 222 include predictions, translations, summaries or media content.

In view of the disclosure above, various examples are set forth below. It should be noted that one or more features of an example, taken in isolation or combination, should be considered within the disclosure of this application.

Example 1 is a method, the method comprising: receiving a natural language query from a user related to a clinico-omics data analysis; performing a context concatenation function combining different information sources to generate an input for a large language model (LLM) agent; processing the natural language query through the LLM agent using at least the input; determining whether additional information is needed after processing the natural language query; performing a tool execution loop when it is determined that additional information is needed; iteratively repeating the tool execution loop until reaching a satisfactory answer or predetermined tool call limit; and generating, after completing the tool execution loop, a final answer and a set of cohorts including a first set of associated SQL queries, a second set of statistical summaries, and a third set of visualization charts.

Example 2 includes the subject matter of Example 1 wherein the tool execution loop comprises: generating tool information, the tool information including tool identification and a tool arguments required for tool execution; selecting a tool, from a set of tools configured for clinico-omics data analysis, using the tool information; executing the selected tool using a tool executor component to generate a tool output; incorporating the tool output to update a conversation history; performing the context concatenation function using at least the updated conversation history to generate an updated input for the LLM agent; processing the natural language query through the LLM agent using at least the updated input; and determining whether other additional information is needed after processing the natural language query, and wherein reaching the satisfactory answer comprises an identification of relevant data fields, appropriate coding values, and necessary dataset metadata.

Example 3 includes the subject matter of any one of Examples 1 and 2, wherein the set of tools includes at least one of a search in descriptor function, a find fields function, a search coding value function, a get coding values function, a search genes function, a search in sequence ontology function, or an evaluate SQL function.

Example 4 includes the subject matter of any one of Examples 1-3, wherein the search in descriptor function is configured to search dataset metadata and descriptive information pertaining to a clinico-omics dataset.

Example 5 includes the subject matter of any one of Examples 1-4, wherein the find fields function is configured to identify relevant data fields within a dataset structure based on semantic analysis of user queries, the search coding value function is configured to search medical coding systems and value mappings, and the get coding values function is configured to retrieve specific coding values and their meanings from medical terminology databases.

Example 6 includes the subject matter of any one of Examples 1-5, wherein the search genes function is configured to search genomic information and gene-related data, the search in sequence ontology function is configured to search biological sequence ontology databases for genomic terminology and classification systems, and the evaluate SQL function is configured to execute and validate SQL queries against a clinico-omics dataset and provide query results for analysis.

Example 7 includes the subject matter of any one of Examples 1-6, further comprising integrating multiple external data sources including a data dictionary storing dataset-specific metadata and field descriptions, reference genome information providing genomic reference data including genes and chromosomes, a sequence ontology database storing biological terminology and classification systems.

Example 8 includes the subject matter of any one of Examples 1-7 wherein the multiple external data sources further comprise an embedding model configured to convert textual information into vector representations for semantic matching, and a vector database configured to store and retrieve vectorized information for semantic searches, wherein the vectorized information enables semantic matching between user query terms and dataset metadata, allowing for identification of relevant fields when exact terminology differs.

Example 9 includes the subject matter of any one of Examples 1-8 wherein generating the final answer and the set of cohorts further comprises processing a set of tool outputs from multiple tools to generate comprehensive analytical results; combining results from genomic searches, field identification, and coding value retrieval to create cohort definitions; and generating statistical summaries and visualization charts.

Example 10 includes the subject matter of any one of Examples 1-9 wherein the LLM agent comprises an assistant application, the assistant application comprising an SQL evaluation engine, a web UI, a clinico-omics data assistant backend, and a database of embeddings.

Example 11 is system comprising: at least one hardware processor; and at least one memory storing instructions that cause the at least one hardware processor to perform operations comprising: receiving a natural language query from a user related to a clinico-omics data analysis; performing a context concatenation function combining different information sources to generate an input for a large language model (LLM) agent; processing the natural language query through the LLM agent using at least the input; determining whether additional information is needed after processing the natural language query; performing a tool execution loop when it is determined that additional information is needed; iteratively repeating the tool execution loop until reaching a satisfactory answer or predetermined tool call limit; and generating, after completing the tool execution loop, a final answer and a set of cohorts including a first set of associated SQL queries, a second set of statistical summaries, and a third set of visualization charts.

Example 12 includes the subject matter of Example 11, wherein the tool execution loop comprises: generating tool information, the tool information including tool identification and a tool arguments required for tool execution; selecting a tool, from a set of tools configured for clinico-omics data analysis, using the tool information; executing the selected tool using a tool executor component to generate a tool output; incorporating the tool output to update a conversation history; performing the context concatenation function using at least the updated conversation history to generate an updated input for the LLM agent; processing the natural language query through the LLM agent using at least the updated input; and determining whether other additional information is needed after processing the natural language query, and wherein reaching the satisfactory answer comprises an identification of relevant data fields, appropriate coding values, and necessary dataset metadata.

Example 13 includes the subject matter of any one of Examples 11-12, wherein the set of tools includes at least one of a search in descriptor function, a find fields function, a search coding value function, a get coding values function, a search genes function, a search in sequence ontology function, or an evaluate SQL function.

Example 14 includes the subject matter of any one of Examples 11-13, wherein the search in descriptor function is configured to search dataset metadata and descriptive information pertaining to a clinico-omics dataset.

Example 15 includes the subject matter of any one of Examples 11-14, wherein the find fields function is configured to identify relevant data fields within a dataset structure based on semantic analysis of user queries, the search coding value function is configured to search medical coding systems and value mappings, and the get coding values function is configured to retrieve specific coding values and their meanings from medical terminology databases.

Example 16 includes the subject matter of any one of 11-15, wherein the search genes function is configured to search genomic information and gene-related data, the search in sequence ontology function is configured to search biological sequence ontology databases for genomic terminology and classification systems, and the evaluate SQL function is configured to execute and validate SQL queries against a clinico-omics dataset and provide query results for analysis.

Example 17 includes the subject matter of any one of 11-16, wherein the operations further comprise integrating multiple external data sources including a data dictionary storing dataset-specific metadata and field descriptions, reference genome information providing genomic reference data including genes and chromosomes, a sequence ontology database storing biological terminology and classification systems.

Example 18 includes the subject matter of any one of 11-17, wherein the multiple external data sources further comprise an embedding model configured to convert textual information into vector representations for semantic matching, and a vector database configured to store and retrieve vectorized information for semantic searches, wherein the vectorized information enables semantic matching between user query terms and dataset metadata, allowing for identification of relevant fields when exact terminology differs.

Example 19 includes the subject matter of any one of 11-18 wherein generating the final answer and the set of cohorts further comprises: processing a set of tool outputs from multiple tools to generate comprehensive analytical results; combining results from genomic searches, field identification, and coding value retrieval to create cohort definitions; and generating statistical summaries and visualization charts.

Example 20 is a non-transitory computer-readable medium comprising instructions that, when executed by at least one processor, configure the at least one processor to perform operations comprising: receiving a natural language query from a user related to a clinico-omics data analysis; performing a context concatenation function combining different information sources to generate an input for a large language model (LLM) agent; processing the natural language query through the LLM agent using at least the input; determining whether additional information is needed after processing the natural language query; performing a tool execution loop when it is determined that additional information is needed; iteratively repeating the tool execution loop until reaching a satisfactory answer or predetermined tool call limit; and generating, after completing the tool execution loop, a final answer and a set of cohorts including a first set of associated SQL queries, a second set of statistical summaries, and a third set of visualization charts.

### Machine Architecture

FIG. 12 is a diagrammatic representation of the machine 1200 within which instructions 1202 (e.g., software, a program, an application, an applet, an app, or other executable code) for causing the machine 1200 to perform any one or more of the methodologies discussed herein may be executed. For example, the instructions 1202 may cause the machine 1200 to execute any one or more of the methods described herein. The instructions 1202 transform the general, non-programmed machine 1200 into a particular machine 1200 programmed to carry out the described and illustrated functions in the manner described. The machine 1200 may operate as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 1202, sequentially or otherwise, that specify actions to be taken by the machine 1200. Further, while a single machine 1200 is illustrated, the term “machine” shall also be taken to include a collection of machines that individually or jointly execute the instructions 1202 to perform any one or more of the methodologies discussed herein. The machine 1200, for example, may comprise the user device 106 or any one of multiple server devices forming part of the server system 104. In some examples, the machine 1200 may also comprise both client and server systems, with certain operations of a particular method or algorithm being performed on the server-side and with certain operations of the particular method or algorithm being performed on the client-side.

The machine 1200 may include processors 1204, memory 1206, and input/output I/O components 1208, which may be configured to communicate with each other via a bus 1210. In an example, the processors 1204 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 1212 and a processor 1214 that execute the instructions 1202. The term “processor” is intended to include multi-core processors that may comprise two or more independent processors (sometimes referred to as “cores”) that may execute instructions contemporaneously. Although FIG. 12 shows multiple processors 1204, the machine 1200 may include a single processor with a single-core, a single processor with multiple cores (e.g., a multi-core processor), multiple processors with a single core, multiple processors with multiples cores, or any combination thereof.

The memory 1206 includes a main memory 1216, a static memory 1218, and a storage unit 1220, both accessible to the processors 1204 via the bus 1210. The main memory 1206, the static memory 1218, and storage unit 1220 store the instructions 1202 embodying any one or more of the methodologies or functions described herein. The instructions 1202 may also reside, completely or partially, within the main memory 1216, within the static memory 1218, within machine-readable medium 1222 within the storage unit 1220, within at least one of the processors 1204 (e.g., within the processor's cache memory), or any suitable combination thereof, during execution thereof by the machine 1200.

The I/O components 1208 may include a wide variety of components to receive input, provide output, produce output, transmit information, exchange information, capture measurements, and so on. The specific I/O components 1208 that are included in a particular machine will depend on the type of machine. For example, portable machines such as mobile phones may include a touch input device or other such input mechanisms, while a headless server machine will likely not include such a touch input device. It will be appreciated that the I/O components 1208 may include many other components that are not shown in FIG. 12. In various examples, the I/O components 1208 may include user output components 1224 and user input components 1226. The user output components 1224 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 1226 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 1208 may include biometric components 1228, motion components 1230, environmental components 1232, or position components 1234, among a wide array of other components. For example, the biometric components 1228 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The biometric components may include a brain-machine interface (BMI) system that allows communication between the brain and an external device or machine. This may be achieved by recording brain activity data, translating this data into a format that can be understood by a computer, and then using the resulting signals to control the device or machine.

Example types of BMI technologies, including:
Electroencephalography (EEG) based BMIs, which record electrical activity in the brain using electrodes placed on the scalp.
Invasive BMIs, which used electrodes that are surgically implanted into the brain.
Optogenetics BMIs, which use light to control the activity of specific nerve cells in the brain.

Any biometric data collected by the biometric components is captured and stored only with user approval and deleted on user request. Further, such biometric data may be used for very limited purposes, such as identification verification. To ensure limited and authorized use of biometric information and other personally identifiable information (PII), access to this data is restricted to authorized personnel only, if at all. Any use of biometric data may strictly be limited to identification verification purposes, and the data is not shared or sold to any third party without the explicit consent of the user. In addition, appropriate technical and organizational measures are implemented to ensure the security and confidentiality of this sensitive information.

The motion components 1230 include acceleration sensor components (e.g., accelerometer), gravitation sensor components, rotation sensor components (e.g., gyroscope).

The environmental components 1232 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

The position components 1234 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1208 further include communication components 1236 operable to couple the machine 1200 to a network 1238 or devices 1240 via respective coupling or connections. For example, the communication components 1236 may include a network interface component or another suitable device to interface with the network 1238. In further examples, the communication components 1236 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1240 may be another machine or any of a wide variety of peripheral devices (e.g., a peripheral device coupled via a USB).

Moreover, the communication components 1236 may detect identifiers or include components operable to detect identifiers. For example, the communication components 1236 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph™, MaxiCode, PDF417, Ultra Code, UCC RSS-2D bar code, and other optical codes), or acoustic detection components (e.g., microphones to identify tagged audio signals). In addition, a variety of information may be derived via the communication components 1236, such as location via Internet Protocol (IP) geolocation, location via Wi-Fi® signal triangulation, location via detecting an NFC beacon signal that may indicate a particular location, and so forth.

The various memories (e.g., main memory 1216, static memory 1218, and memory of the processors 1204) and storage unit 1220 may store one or more sets of instructions and data structures (e.g., software) embodying or used by any one or more of the methodologies or functions described herein. These instructions (e.g., the instructions 1202), when executed by processors 1204, cause various operations to implement the disclosed examples.

The instructions 1202 may be transmitted or received over the network 1238, using a transmission medium, via a network interface device (e.g., a network interface component included in the communication components 1236) and using any one of several well-known transfer protocols (e.g., hypertext transfer protocol (HTTP)). Similarly, the instructions 1202 may be transmitted or received using a transmission medium via a coupling (e.g., a peer-to-peer coupling) to the devices 1240.

The terms “machine-readable medium,” “computer-readable medium,” and “device-readable medium” mean the same thing and may be used interchangeably in this disclosure. The terms are defined to include both machine-storage media and transmission media. Thus, the terms include both storage devices/media and carrier waves/modulated data signals.

The various operations of example methods described herein may be performed, at least partially, by one or more processors that are temporarily configured (e.g., by software) or permanently configured to perform the relevant operations. Similarly, the methods described herein may be at least partially processor implemented. For example, at least some of the operations of a method may be performed by one or more processors. The performance of certain of the operations may be distributed among the one or more processors, not only residing within a single machine, but also deployed across a number of machines. In some example embodiments, the processor or processors may be in a single location (e.g., within a home environment, an office environment, or a server farm), while in other embodiments the processors may be distributed across a number of locations.

Although the embodiments of the present disclosure have been described with reference to specific example embodiments, it will be evident that various modifications and changes may be made to these embodiments without departing from the broader scope of the inventive subject matter. Accordingly, the specification and drawings are to be regarded in an illustrative rather than a restrictive sense. The accompanying drawings that form a part hereof show, by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Thus, although specific embodiments have been illustrated and described herein, it should be appreciated that any arrangement calculated to achieve the same purpose may be substituted for the specific embodiments shown. This disclosure is intended to cover all adaptations or variations of various embodiments. Combinations of the above embodiments, and other embodiments not specifically described herein, will be apparent to those of skill in the art, upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim is still deemed to fall within the scope of that claim.

What is claimed is:

1. A method comprising:
receiving a natural language query from a user related to a clinico-omics data analysis;
performing a context concatenation function combining different information sources to generate an input for a large language model (LLM) agent;
processing the natural language query through the LLM agent using at least the input;
determining whether additional information is needed after processing the natural language query;
performing a tool execution loop when it is determined that additional information is needed;
iteratively repeating the tool execution loop until reaching a satisfactory answer or predetermined tool call limit; and
generating, after completing the tool execution loop, a final answer and a set of cohorts including a first set of associated SQL queries, a second set of statistical summaries, and a third set of visualization charts.

2. The method of claim 1, wherein the tool execution loop comprising:
generating tool information, the tool information including tool identification and a tool arguments required for tool execution;
selecting a tool, from a set of tools configured for clinico-omics data analysis, using the tool information;
executing the selected tool using a tool executor component to generate a tool output;
incorporating the tool output to update a conversation history;
performing the context concatenation function using at least the updated conversation history to generate an updated input for the LLM agent;
processing the natural language query through the LLM agent using at least the updated input; and
determining whether other additional information is needed after processing the natural language query, and
wherein reaching the satisfactory answer comprises an identification of relevant data fields, appropriate coding values, and necessary dataset metadata.

3. The method of claim 2, wherein the set of tools includes at least one of a search in descriptor function, a find fields function, a search coding value function, a get coding values function, a search genes function, a search in sequence ontology function, or an evaluate SQL function.

4. The method of claim 3, wherein the search in descriptor function is configured to search dataset metadata and descriptive information pertaining to a clinico-omics dataset.

5. The method of claim 3, wherein the find fields function is configured to identify relevant data fields within a dataset structure based on semantic analysis of user queries, the search coding value function is configured to search medical coding systems and value mappings, and the get coding values function is configured to retrieve specific coding values and their meanings from medical terminology databases.

6. The method of claim 3, wherein the search genes function is configured to search genomic information and gene-related data, the search in sequence ontology function is configured to search biological sequence ontology databases for genomic terminology and classification systems, and the evaluate SQL function is configured to execute and validate SQL queries against a clinico-omics dataset and provide query results for analysis.

7. The method of claim 1, further comprising integrating multiple external data sources including a data dictionary storing dataset-specific metadata and field descriptions, reference genome information providing genomic reference data including genes and chromosomes, a sequence ontology database storing biological terminology and classification systems.

8. The method of claim 7, wherein the multiple external data sources further comprise an embedding model configured to convert textual information into vector representations for semantic matching, and a vector database configured to store and retrieve vectorized information for semantic searches, wherein the vectorized information enables semantic matching between user query terms and dataset metadata, allowing for identification of relevant fields when exact terminology differs.

9. The method of claim 1, wherein generating the final answer and the set of cohorts further comprises:
processing a set of tool outputs from multiple tools to generate comprehensive analytical results;
combining results from genomic searches, field identification, and coding value retrieval to create cohort definitions; and
generating statistical summaries and visualization charts.

10. The method of claim 1, wherein the LLM agent comprises an assistant application, the assistant application comprising an SQL evaluation engine, a web UI, a clinico-omics data assistant backend, and a database of embeddings.

11. A system comprising:
at least one hardware processor; and
at least one memory storing instructions that cause the at least one hardware processor to perform operations comprising:
receiving a natural language query from a user related to a clinico-omics data analysis;
performing a context concatenation function combining different information sources to generate an input for a large language model (LLM) agent;
processing the natural language query through the LLM agent using at least the input;
determining whether additional information is needed after processing the natural language query;
performing a tool execution loop when it is determined that additional information is needed;
iteratively repeating the tool execution loop until reaching a satisfactory answer or predetermined tool call limit; and
generating, after completing the tool execution loop, a final answer and a set of cohorts including a first set of associated SQL queries, a second set of statistical summaries, and a third set of visualization charts.

12. The system of claim 11, wherein the tool execution loop comprises:
generating tool information, the tool information including tool identification and a tool arguments required for tool execution;
selecting a tool, from a set of tools configured for clinico-omics data analysis, using the tool information;
executing the selected tool using a tool executor component to generate a tool output;
incorporating the tool output to update a conversation history;
performing the context concatenation function using at least the updated conversation history to generate an updated input for the LLM agent;
processing the natural language query through the LLM agent using at least the updated input; and
determining whether other additional information is needed after processing the natural language query, and
wherein reaching the satisfactory answer comprises an identification of relevant data fields, appropriate coding values, and necessary dataset metadata.

13. The system of claim 12, wherein the set of tools includes at least one of a search in descriptor function, a find fields function, a search coding value function, a get coding values function, a search genes function, a search in sequence ontology function, or an evaluate SQL function.

14. The system of claim 13, wherein the search in descriptor function is configured to search dataset metadata and descriptive information pertaining to a clinico-omics dataset.

15. The system of claim 13, wherein the find fields function is configured to identify relevant data fields within a dataset structure based on semantic analysis of user queries, the search coding value function is configured to search medical coding systems and value mappings, and the get coding values function is configured to retrieve specific coding values and their meanings from medical terminology databases.

16. The system of claim 13, wherein the search genes function is configured to search genomic information and gene-related data, the search in sequence ontology function is configured to search biological sequence ontology databases for genomic terminology and classification systems, and the evaluate SQL function is configured to execute and validate SQL queries against a clinico-omics dataset and provide query results for analysis.

17. The system of claim 11, wherein the operations further comprise integrating multiple external data sources including a data dictionary storing dataset-specific metadata and field descriptions, reference genome information providing genomic reference data including genes and chromosomes, a sequence ontology database storing biological terminology and classification systems.

18. The system of claim 17, wherein the multiple external data sources further comprise an embedding model configured to convert textual information into vector representations for semantic matching, and a vector database configured to store and retrieve vectorized information for semantic searches, wherein the vectorized information enables semantic matching between user query terms and dataset metadata, allowing for identification of relevant fields when exact terminology differs.

19. The system of claim 11, wherein generating the final answer and the set of cohorts further comprises:
processing a set of tool outputs from multiple tools to generate comprehensive analytical results;
combining results from genomic searches, field identification, and coding value retrieval to create cohort definitions; and
generating statistical summaries and visualization charts.

20. A non-transitory computer-storage medium comprising instructions that, when executed by one or more processors of a machine, configure the machine to perform operations comprising:
receiving a natural language query from a user related to a clinico-omics data analysis;
performing a context concatenation function combining different information sources to generate an input for a large language model (LLM) agent;
processing the natural language query through the LLM agent using at least the input;
determining whether additional information is needed after processing the natural language query;
performing a tool execution loop when it is determined that additional information is needed;
iteratively repeating the tool execution loop until reaching a satisfactory answer or predetermined tool call limit; and
generating, after completing the tool execution loop, a final answer and a set of cohorts including a first set of associated SQL queries, a second set of statistical summaries, and a third set of visualization charts.
